(19) United States
(12) Patent Application Publication
(10) Pub. No.: US 2012/0264872 A1
(43) Pub. Date: Oct. 18, 2012

(54) LCST POLYMERS

(75) Inventors: Thomas Weiss, Ilvesheim (DE); Hans-Peter Seelmann-Eggebert, Limburgerhof (DE); Andrea Misske, Speyer (DE); Wolfgang Spiegler, Worms (DE)

(73) Assignee: BASF SE, Ludwigshafen (DE)

(21) Appl. No.: 13/438,289

(22) Filed: Apr. 3, 2012

Related U.S. Application Data
(60) Provisional application No. 61/474,332, filed on Apr. 12, 2011.

(51) Int. Cl. C08F 36/20; C09D 133/08; B05D 3/02; B05D 7/06; B05D 7/02; C03C 17/32; B05D 7/14; C07C 69/533; B05D 1/02

(52) U.S. Cl. 524/558; 560/224; 427/385.5; 427/393.5; 427/389.7; 427/388.1; 427/393

(57) ABSTRACT

The invention relates to LCST polymers and their preparation and use.

This invention relates to LCST polymers and to their preparation and use.

So-called LCST polymers based on polyalkylene oxides are known. LCST is short for lower critical solution temperature, and an LCST polymer is a polymer which is soluble in a liquid medium at a lower temperature but precipitates from the liquid medium above a certain temperature, namely the cloud point or the LCST temperature. This process is reversible, so the system becomes homogeneous again on cooling down. The temperature at which the solution clarifies on cooling down is known as the cloud point (see German standard specification DIN EN 1890 of September 2006). This temperature is characteristic for a particular substance.

WO 01/60926 A1 discloses a process for coating particles with LCST polymers wherein the LCST polymer is dissolved in a solvent at below the LCST, the solution obtained is mixed with the particles to be coated, and then the temperature of the mixture obtained is raised to a temperature above the LCST to precipitate the LCST polymers on the particle surfaces.

WO 2004/046258 A2 discloses LCST polymers based on polyalkylene oxides terminally substituted with an optionally substituted acrylate. WO 2004/046258 A2 further discloses using these LCST polymers for coating particles and non-particulate substrate surfaces by contacting the polymers in a liquid medium with the particles and surfaces, respectively, at below the LCST temperature, raising the temperature to above the LCST temperature and polymerizing the polymers on the surface of the particles, and on the surfaces, respectively, via the double bonds at this or a higher temperature.

The invention provides compounds, more particularly LCST Polymers, of the general formula I

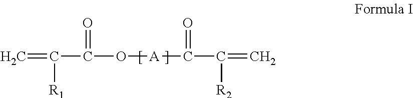

where

R₁ and R₂ are each (independently) hydrogen or an alkyl radical, more particularly of 1-4
carbon atoms, more particularly methyl,

A is a radical comprising the radicals A1 and A2,

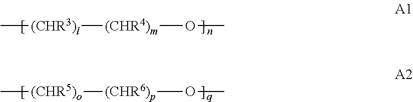

where

R³ to R⁶ are the same or different and are each H, C1— to C5-alkyl, more particularly methyl, ethyl, propyl or aryl, more particularly phenyl,

l is 1 or 0,

m is 1 or 0 provided l+m is at least 1,

n is from 1 to 100,

o is from 0 to 5,

p is from 0 to 5 provided o+p is from 3 to 5,

q is from 1 to 100,

wherein said compound of formula I, when measured according to German standard specification DIN EN 1890 of September 2006, procedure as per method E, has a cloud point in the range from 40 to 80° C. and more particularly in the range from 60 to 80° C., and the A2 units are comprised in the A radical in the terminal position.

According to the procedure as per method E of German standard specification DIN EN 1890 of September 2006, 5 g of sample were introduced into an Erlenmeyer flask and admixed with 25 g of the aqueous solution of diethylene glycol mono-n-butyl ether (BDG) having a mass fraction of 25%. The mixture is stirred until the sample has formed a clear solution. A heating device is then used to heat the liquid under slow agitation until it is completely cloudy. This is followed by gradual cooling under agitation. The temperature at which the cloudiness disappears, i.e., the solution clarifies, is the cloud point.

A1 is more particularly one of the following radicals:

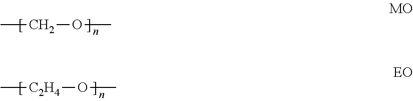

A2 is more particularly one of the following radicals:

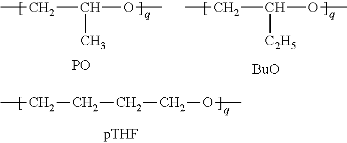

where n and q are each as defined above.

A preferably consists of radicals A1 and A2 and more particularly is the group -A2-A1-A2-.

The recurring units in the radicals A1 and A2 may form a random distribution or a blockwise arrangement.

When the radical A2 includes not only propylene glycol radicals but also polytetrahydrofuran units, it is a preferred embodiment for the polytetrahydrofuran units to be terminally disposed.

The radicals A1 and A2 may be substituted, more particularly with an alkyl radical of 1-4 carbon atoms or an aryl radical, more particularly phenyl. An example of a substituted recur- ring radical is the phenyl-substituted ethylene glycol radical (styrene oxide).

In a particularly preferred embodiment, A has the structure

-(PO)ₓ-(EO)_{y}-(PO)_{z}-,

where

EO is ethylene glycol radical,

PO is propylene glycol radical,

x and z are the same or different and are each from 1 to 100 and more particularly from 1 to
20, and

y is from 1 to 100 and more particularly from 1 to 90.

In a further preferred embodiment,

x and z are the same or different and are each from 5 to 10, and

y is from 60 to 80 and more particularly from 65 to 75.

The structure -(PO)ₓ-(EO)_{y}-(PO)_{z} preferably has an EO weight fraction of 5% to 85% by weight and a number average molar mass (Mn) in the range from 200 to 50 000 g/mol.

Particularly preferred compounds conform to the following formula:

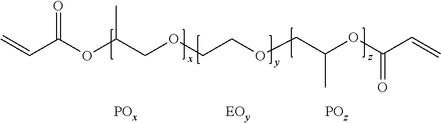

where

x and z are the same or different and range from 0.1 to 200 and more particularly from 5 to 20,
and

y in each occurrence is the same or different and in the range from 1 to 100 and more
particularly from 60 to 80.

The compounds according to the invention are very useful for producing coatings by applying the compounds to the surface of substrates and polymerizing.

The invention accordingly further provides a process for producing coatings based on a compound of formula I on the surface of a formed article from a meltable substrate S, more particularly on fibers in a melt-spinning process or self-supporting films after extrusion, which process comprises

a) providing the substrate in molten form,

b) forming the molten substrate via a suitable device V, preferably a die or slot, into a formed article, more particularly into a fiber or self-supporting film, wherein

c) the formed article has a temperature above the cloud point of said compound of formula I on emergence from said device V, and

d) contacting the surface of the formed article above the cloud point with said compound of formula I to deposit said compound of formula I,

wherein the polymerization of the double bonds of said compound of formula I is initiated after deposition on the surface to form a preferably crosslinked coating on the surface.

In a preferred embodiment
the formed article after emergence from said device V is led into a preferably aqueous bath B comprising said compound of formula I and optionally a polymerization initiator, wherein the formed article has a temperature above the cloud point as it enters the bath,
said compound of formula I deposits on the formed article,
the bath has a temperature below the cloud point, and
the polymerization is performed at a temperature below the cloud point.

In a further embodiment, the invention provides a process for producing coatings on the surface of a formed article from a meltable substrate S, more particularly on fibers, in a melt-spinning process or self-supporting films after extrusion as per the above-recited steps a) to d), wherein after deposition of said compound of formula I according to the present invention on the surface the polymerization of the double bonds of said compound of formula I is performed by photopolymerization. Photopolymerization can be induced by irradiating the system with electromagnetic radiation of a wavelength which is directly absorbed by the compounds of formula I, or polymerization is induced by irradiation using a photoinitiator.

In a further preferred embodiment, the formed article emerging from said device V is immediately thereafter led at a temperature above the cloud point into a spraying device and is sprayed therein with said compound of formula I, preferably in the form of an aqueous solution, wherein the temperature of the spray-dispensed polymer is below the cloud point, to deposit said compound of formula 1 on the surface of the substrate.

Particularly preferred substrates are polyamides, polyesters, polypropylene or polyurethanes. In a further preferred embodiment, the substrate is a glass, steel or wood.

The polymerization of the double bonds can be performed in bath B or subsequently to the treatment in bath B. To initiate the polymerization, it is preferable to add a polymerization initiator.

The polymerization of the acrylate groups of the compounds of formula 1 according to the present invention is preferably carried out free-radically, more particularly from an aqueous or alcoholic solution. The polymerization is preferably carried out in the presence of free-radical formers, more particularly organic or inorganic peroxides, azo compounds or metals/organometallic compounds. The molar mass of the polymers obtained may be controlled by adding suitable chain transfer agents, more particularly mercaptans, organic halogen compounds, aldehydes or xanthates or nitroxyl free radical formers. The polymerization temperature is preferably in the range from 50 to 100° C. and more particularly in the range from 60 to 80° C.

The polymerization of the acrylate groups of the compounds of formula I according to the present invention can also be initiated using a photoinitiator. Photoinitiator quantities used are generally in the range from 0.01% to 10% by weight and more particularly from 0.01% to 3% by weight, all based on the compound of formula I. The compounds useful as photoinitiators are capable on exposure to actinic light of forming free radicals and of inducing a rapid photopolymerization of the compound of formula I. Possible photoinitiators include, for example, acyloins and acyloin ethers, aromatic diketones and their derivatives and polynuclear quinones. Of particular suitability are benzoin and alpha-hydroxymethylbenzoin methyl ether or benzoin methyl ether, benzoin isopropyl ether, benzil monoketals such as benzil dimethyl ketal, benzil methyl ethyl ketal, benzil methyl benzyl ketal, benzil neopentyl ketal or diaryiphosphine oxides as described in German Laid-Open Specification DOS 29 09 992, preferably 2,6-dimethoxybenzoyldiphenylphosphine oxide and more particularly 2,4,6-trimethyl-benzoyldiphenylphosphine oxide. Preference is given to photoinitiators in terms of type and amount such that they need only short minimum exposure times, preferably not more than a few minutes, to initiate the photopolymerization on imagewise exposure to actinic light, more particularly UV light.

When photoinitiators are used, there may in addition also be used inhibitors of thermal polymerization, such as hydroquinone, p-methoxyphenol, dinitrobenzene, p-quinone, methylene blue, beta-naphthol, N-nitrosamines such as N-nitrosodiphenylamine, phenothiazine, phosphorous esters such as triphenyl phosphite or the salts and more particularly the alkali metal and aluminum salts of N-nitrosocyclohexylhydroxylamine. The inhibitors can be used in amounts of 0.001% to 3% and preferably 0.01% to 1% by weight, based on the compound of formula I.

In a further embodiment, the acrylate end groups in the compounds of formula I which are to be used according to the present invention can be replaced in an amount from 0.1 to 99 mol %, more particularly 20 to 50 mol %, by radicals of

a) maleic acid or maleic acid derivatives (particularly esters)

b) fumaric acid and esters

c) hydroxyalkyl acrylates

d) vinyl ethers

e) glycidyl (meth)acrylate

f) allyl glycidyl ethers

g) hydroxybutyl vinyl ethers

Preparing the compounds of formula I

In a preferred embodiment, the inventive compounds of formula I

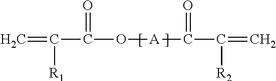

are prepared by reacting a compound of the general formula

HO—AOH

where A is as defined above, with a compound of the formula

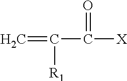

and optionally

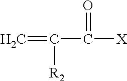

where

R1 and R2 are each as defined above, and

X is hydroxyl, halogen, preferably chlorine, an acid group, an alkyl group or an alkoxy group of 1 to 100 carbon atoms, in a molar ratio of at least 1:1 to 1:4 and more particularly 1:2.

The reaction is preferably performed in a solvent, such as tertiary monools, preferably tert-butanol, tert-amyl alcohol, pyridine, poly-C1-C4-alkylene glycol di-C1-C4-alkyl ethers, preferably polyethylene glycol di-C1-C4-alkyl ethers, e.g., 1,2-dimethoxyethane, diethylene glycol dimethyl ether, polyethylene glycol dimethyl ether 500, methyl tert-butyl ether, ethyl tert-butyl ether, C1-C4-alkylene carbonates, more particularly propylene carbonate, C3-C6-alkyl acetates, more particularly tert-butyl acetate, tetrahydrofuran, toluene, 1,3-dioxolane, acetone, isobutyl methyl ketone, ethyl methyl ketone, 1,4-dioxane, tert-butyl methyl ether, cyclohexane, methylcyclohexane, toluene, hexane, dimethoxymethane, 1,1-dimethoxyethane, acetonitrile, and also mono- or multi-phasic mixtures thereof.

It can be advantageous to remove liberated water but more particularly to perform the reaction without solvent, i.e., in the acrylic acid derivatives themselves, particularly at a temperature of 20 to 200° C. in the presence of suitable chemical catalysts or biological enzymes, preferably at a pH of 2 to 11.

Preferred compounds of formula I according to the invention are obtainable by

1. direct reaction of preformed compounds

with (meth)acrylic acid or (meth)acrylic acid derivatives

HO—AOH

with (meth)acrylic acid or (meth)acrylic acid derivatives

2. by transesterification of acrylic esters and methacrylic esters under
a) chemical
b) enzymatic catalysis.

Useful acrylic or methacrylic esters for transesterification include for example: alkyl (meth)acrylates of straight-chain, branched or cycloaliphatic alcohols having 1 to 40 carbon atoms, for example methyl (meth)acrylate, ethyl (meth)acrylate, n-butyl (meth)acrylate, i-butyl (meth)acrylate, t-butyl (meth)acrylate, pentyl (meth)acrylate, 2-ethylhexyl (meth)acrylate, stearyl (meth)acrylate, lauryl (meth)acrylate, cyclohexyl (meth)acrylate, isobornyl (meth)acrylate; aryl (meth)acrylates such as, for example, benzyl (meth)acrylate or phenyl (meth)acrylate, which may each be unsubstituted aryl radicals or aryl radicals substituted 1-4 times; other aromatically substituted (meth) acrylates such as, for example, naphthyl (meth)acrylate; mono(meth)acrylates of ethers, polyethylene glycols, polypropylene glycols or mixtures thereof with 5-80 carbon atoms, for example tetrahydrofurfuryl methacrylate, methoxy(m)ethoxyethyl methacrylate, 1-butoxypropyl methacrylate, cyclohexyloxymethyl methacrylate, benzyloxymethyl methacrylate, furfuryl methacrylate, 2-butoxyethyl methacrylate, 2-ethoxyethyl methacrylate, allyloxymethyl methacrylate, 1-ethoxybutyl methacrylate, 1-ethoxyethyl methacrylate, ethoxymethyl methacrylate, poly(ethylene glycol) methyl ether (meth)acrylate and poly(propylene glycol) methyl ether (meth)acrylate.

Preferred parameters for a direct synthesis are:

temperature: 80-160° C., preferably 90-130° C.

alcohol/(meth)acrylic acid: 1:0.7-1.2 (molar)

catalyst: sulfuric acid or sulfonic acids, preferably methanesulfonic acids

catalyst quantity: 0.1-10% by weight (preferably 0.5-5% by weight) based on
starting materials

reaction time: 1-10 h, preferably 1-6 h

Optionally, an entraining agent (e.g., cyclohexane or toluene) is used to remove the water of esterification. The esterification can be performed under atmospheric pressure, under superatmospheric pressure or under reduced pressure not only continuously but also batchwise.

Preferred parameters and starting materials for a transesterification are:

temperature: 30-180° C., preferably 50-130° C.

catalyst quantity: 0.01 to 10% by weight of catalyst, preferably from 0.1% to 5% by weight,
more preferably from 0.2% to 2% by weight of catalyst, based on the
entire reaction mixture

catalysts: organometal oxides, organometal halides such as diorganotin oxides,
diorganotin halides, alkali metal salts of inorganic acid especially of
phosphoric acid, transition metal alkoxides such as titanium alkoxides,
alkali amides such as lithium amide, alkali metal and alkaline earth
metal alkoxides such as potassium tert-butoxide, acids such as sulfuric
acid, alkyl- or arylsulfonic acids e.g., p-toluenesulfonic acid and
methane-sulfonic acid, inorganic acid or basic (mixed) oxides such as zeolites,
aluminum-silicon mixed oxide, titanium-silicon mixed oxide or
magnesium oxide, or magnesium silicates

with and without solvent in excesses of (meth)acrylic esters of 1:50 to 1:500 mol/mol, more preferably 1:100 to 1:400 based on the substrate.

A chemical transesterification can be carried out under atmospheric pressure, under superatmospheric pressure or under reduced pressure not only continuously but also batchwise.

An enzymatically catalyzed transesterification is preferably carried out under the following conditions:

temperature: 10-80° C., more preferably at 20-40° C.

pH: 5-8

catalyst: lipases

with and without solvent in excesses of (meth)acrylic esters of 1:50 to 1:500 mol/mol, more preferably 1:100 to 1:400 based on the substrate.

An enzymatic transesterification can be carried out under atmospheric pressure, under superatmospheric pressure or under reduced pressure not only continuously but also batchwise.

Preferred devices V are for example

a) the devices known in connection with melt spinning of fibers.

They include, for the process of the invention, depending on the embodiment, either a spraying device immediately following the emergence of fiber at the die, or a bath which preferably comprises an aqueous solution of the polymers according to the invention. This bath may further comprise a polymerization initiator to conduct the polymerization. However, it is also possible to apply the polymerization initiator to the fiber in an additional further bath. This is more particularly advantageous to avoid any premature polymerization of the polymer in the coagulation bath.

b) the conventional film production rigs, for example based on an extruder which conveys the melt of the substrate and/or melts the substrate, and extrudes it through a die, more particularly a wide slot die, to form a film.

In a further preferred embodiment, effect agents are deposited on the formed body together with the compounds of formula I according to the invention. Effect agents are more particularly compounds to improve the properties of the formed body, more particularly of the films and fibers, for example UV stabilizers, pigments, nanoparticles, IR-absorbing compounds, etc. For this, the effect agents can preferably be comprised in bath B together with the polymers.

The formed articles of the present invention, i.e., films and fibers, can be drawn in a conventional manner, including more particularly after application and polymerization of the acrylate groups.

### EXAMPLES

### Example 1.1

### Synthesis of Polyethylene Glycol-Polypropylene Glycol Copolymers

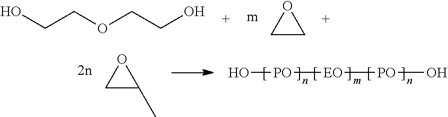

A cleaned 5 l steel reactor was initially charged with diethylene glycol (53.05 g, 0.5 mol) and potassium t-butoxide (10.2 g, 0.5% by weight of final quantity). The system was inertized three times with nitrogen to 5 bar and heated to 120° C. at which point ethylene oxide was added by mass-controlled metering (1364.0 g). The temperature was then raised to 130° C. and propylene oxide (638 g, 11 mol) was added in the course of 700 min. The system was subsequently allowed to react for 4 h and cooled down to 80° C. with stirring.

The crude product was admixed with magnesium silicate (3% by weight) and, after 1 h of stirring on a rotary evaporator, pressure filtered through a 900 Seitz filter medium.

The product was characterized by determining its cloud point using method E of EN 1890: 5 g of sample were dissolved in 25 g of aqueous butyldiglycol solution (c=250 g/l) and heated.

Yield: 2101.33 g

Mn: 4700

Mw: 5000

Cloud point: 71 ° C.

### Example 1.2

### Synthesis of (Polyethylene Glycol-Polypropylene Glycol) Diacrylate:

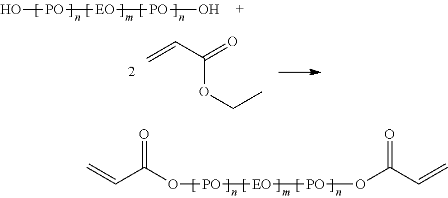

The following were combined in a 6 l four-neck flask: PPG-PEG-PPG (250 g; 0.05 mol) from example 1.1, ethyl acrylate (1000 g; 10 mol), molecular sieve 5 A powder (Fluka) 55.5 g (10 times the amount based on theoretical amount of alcohol formed).

The following were added as stabilizers: 4-methoxyphenol (MeHQ): 98.6 mg; (Aldrich) (99% [GC]) (400 ppm based on theoretic amount of product); phenothiazine (PTZ): 3.0 mg (Fluka) (purum; ≧98.0% [GC]) (10 ppm based on theoretical amount of product).

Novozym 435 (33.71 g) (7.0% by weight based on starting material) was added at about 30° C. during the heating phase. The batch was stirred for 48 h at 40° C. (bath 46° C.) and 150 rpm using a glass PTFE intensive stirrer with air introduction.

The batch was then filtered off with suction through a 4 l glass filter nutsche G 2 filled with silica gel 60 (0.040-0.064 mm) at about 500-600 mbar, washed with 4 l of acetone and dried at 60° C. in a rotary evaporator with air introduction to remove ethyl acrylate residues.

The product was characterized by determining its cloud point using method E of EN 1890: 5 g of sample were dissolved in 25 g of aqueous butyldiglycol solution (c=250 g/l) and heated.

Yield: 260 g of clear oil, solidifies at room temperature

Mn: 4900

Mw: 5200

Cloud point: 66° C.

### Example 2

### Fiber Coating

Fiber coating was tested using a melt-spinning process for polypropylene as an example. An aqueous solution of the following components (% by weight) was used for this purpose:

10% of LCST polymer as per example 1.3

0.5% of ethylenediaminetetraacetic acid sodium salt 0.1% of adhesion promoter (polyethyleneimine) 0.5% of 2,2′-azobis[2-(2-imidazolin-2yl)propane] dihydrochloride free-radical initiator

Balance: water

A two-stage extrusion spinning rig (POY, FDY) is used to obtain microfibers (120 dtexf32) [dtex: is a unit of measurement for the fineness of threads. It indicates the weight in g of 10 000 m of fiber] from a melt at a die temperature of 295° C. In the course of the cooling profile, the fiber was contacted with the LCST polymer solution at a temperature of 80-100° C. (about 100 cm after emergence of the fiber material from the spinneret die) by spraying. The fiber-coated yarns obtained had improved properties, particularly in respect of flexibility and affinity.

We claim:

1. A compound of the general formula I 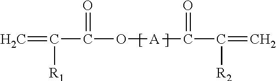
where
R₁ and R₂ are each (independently) hydrogen or an alkyl radical, more particularly
methyl,
A is a radical comprising the radicals A1 and A2, 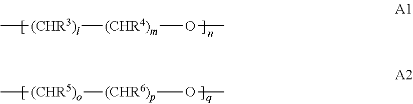
where
R³ to R⁶ are the same or different and are each H, C1- to C5-alkyl, more particularly methyl, ethyl, propyl or aryl, more particularly phenyl,
l is 1 or 0,
m is 1 or 0 provided l+m is at least 1,
n is from 1 to 100,
o is from 0 to 5,
p is from 0 to 5 provided o+p is from 3 to 5,
q is from 1 to 100,
wherein said compound of formula I, when measured according to German standard specification DIN EN 1890 of September 2006, procedure as per method E, has a cloud point in the range from 40 to 80° C. and more particularly in the range from 60 to 80° C., and the A2 units are comprised in the A radical in the terminal position.

2. The compound according to claim 1 wherein A consists of A1 and A2.

3. The compound according to claim 1 wherein A1 is one of the following radicals:

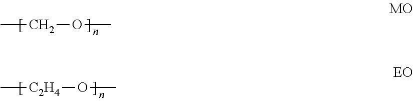

4. The compound according to claim 1 wherein A2 is one of the following radicals:

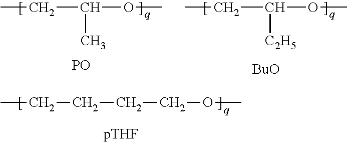

5. The compound according to claim 1 wherein A has one of the following structures:
(PO)ₓ-(EO)_{y}(PO)_{z}
or
-(pTHF-)ₓ-(EO)_{y}-(pTHF)_{z}-,
where EO, PO and pTHF are each as defined in claims 2 and 3 and where
x and z are the same or different and are each from 1 to 100 and more particularly
from 1 to 10 and
y is from 1 to 100 and more particularly from 10 to 90.

6. The compound according to claim 5 wherein
x and z are the same or different and are each from 5 to 20, and
y is from 60 to 80 and more particularly from 65 to 75.

7. The method of using the compound according to claim 1 for producing coatings by applying the compound to the surface of a substrate and polymerizing.

8. A process for producing coatings based on a compound of formula I according to claim 1 on the surface of a formed article from a meltable substrate S, more particularly on fibers in a melt-spinning process or self-supporting films after extrusion, which process comprises
a) providing the substrate in molten form,
b) forming the molten substrate via a suitable device V, preferably a die or slot, into a formed article, more particularly into a fiber or self-supporting film, wherein
c) the formed article has a temperature above the cloud point of said compound of formula I on emergence from said device V, and
d) contacting the surface of the formed article above the cloud point with said compound of formula Ito deposit said compound of formula I, wherein the polymerization of the double bonds of said compound of formula I is initiated after deposition on the surface to form a preferably crosslinked coating on the surface.

9. The process according to claim 8 wherein
the formed article after emergence from said device V is led into a preferably aqueous bath B comprising said compound of formula I and optionally a polymerization initiator, wherein the formed article has a temperature above the cloud point as it enters the bath,
said compound of formula I deposits on the formed article,
the bath has a temperature below the cloud point, and
the polymerization is performed at a temperature below the cloud point, or wherein
after deposition of said compound of formula I according to the present invention on the surface the polymerization of the double bonds of said compound of formula I is performed by photopolymerization.

10. The process according to claim 8 wherein the formed article emerging from said device V is immediately thereafter led at a temperature above the cloud point into a spraying device and is sprayed therein with said compound of formula I, preferably in the form of an aqueous solution, wherein the temperature of the spray-dispensed polymer is below the cloud point, to deposit said compound on the surface of the substrate.

11. The process according to claim 8 wherein the substrate is a polyamide, polyester, polypropylene or polyurethane.

12. The process according to claim 8 wherein the formed article after coating is drawn and wound up.

13. The process according to claim 8 wherein the substrate is a glass, steel or wood.

14. The process according to claim 9 wherein the polymerization of the double bonds is performed subsequently to the treatment in bath B.

15. A formed article obtainable according to claim 8.
